(19) United States
(12) Patent Application Publication
(10) Pub. No.: US 2025/0222245 A1
(43) Pub. Date: Jul. 10, 2025

(54) SEMI-PERMANENT TATTOO TRANSFER DEVICES AND IMAGE-FORMING COMPOSITIONS

(71) Applicant: TMI Acquisition, LLC, Tucson, AZ (US)

(72) Inventors: Christopher Huff, Tucson, AZ (US); Cynthia Hiroko Tanaka, Tucson, AZ (US)

(21) Appl. No.: 19/012,676

(22) Filed: Jan. 7, 2025

Related U.S. Application Data
(60) Provisional application No. 63/618,765, filed on Jan. 8, 2024.
(60) Provisional application No. 63/557,305, filed on Feb. 23, 2024.

(51) Int. Cl. A61M 37/00; B41M 3/00; B41M 3/12; C09B 61/00

(52) U.S. Cl. CPC A61M 37/0076; B41M 3/006; B41M 3/12; C09B 61/00

(57) ABSTRACT

Described herein are semi-permanent tattoo transfer devices or structures for applying semi-permanent tattoo images to the skin of human and animal subjects, and image-forming compositions used in the devices. The devices are used to apply a flexible image-forming composition layer to an human or animal subject's skin, wherein the image-forming composition layer is comfortable for the subject to wear and will eventually slough off with normal skin cell turnover of the subject's skin. Within about 24-36 hours after application to the subject's skin, an image-forming component (such as Genipin) in the image-forming composition layer causes development of a tattoo image on the skin, due to interactions with the skin or components of the skin. The devices may also be used to apply an image-forming composition that dyes the skin (such as Henna) instead of reacting with the skin to color it as does Genipin. Use of the devices results in the formation of a semi-permanent tattoo image on the subject's skin, wherein the tattoo image lasts for one to two weeks and is semi-permanent, meaning that the tattoo image cannot be removed by washing or even scrubbing the subject's skin.

### CROSS-REFERENCE TO RELATED APPLICATIONS

This application claims priority to U.S. provisional application No. 63/618,765 filed on Jan. 8, 2024 and U.S. provisional application No. 63/557,305 filed on Feb. 23, 2024, both of which are hereby incorporated herein by reference for all that they each disclose.

### FIELD OF THE INVENTION

The present invention is in the field of semi-permanent tattoos for application to the skin of humans and animals.

### BACKGROUND OF THE INVENTION

The invention relates to the manufacture of semi-permanent tattoos, for application to human and animal skin, primarily but not necessarily only for decorative purposes.

### SUMMARY OF THE INVENTION

Described herein are semi-permanent tattoo transfer devices or structures for applying semi-permanent tattoos, and the image-forming compositions used in the devices. The devices described herein are used to apply an image-forming composition layer or film to a human or animal subject's skin. Once applied to the subject's skin, the image-forming composition layer is flexible and comfortable to the subject, and will eventually slough off with normal skin cell turnover of the subject's skin. Use of the devices results in the formation of a clearly defined semi-permanent tattoo image on the subject's skin, wherein the tattoo image lasts for one to two weeks and cannot be removed by washing or even scrubbing the subject's skin.

### DETAILED DESCRIPTION OF THE INVENTION

The terms “semi-permanent tattoo transfer device” and “semi-permanent tattoo transfer structure” are referred to herein as “device” or “devices”, and refer to multi-layer structures described herein that are used to transfer at least one layer of an image-forming composition onto a human or animal subject's skin, to produce a semi-permanent image (also referred to herein as a “semi-permanent tattoo”, “semi-permanent tattoo image”, or “tattoo image”) on the subject's skin. The tattoo image can be of virtually any design. For example the tattoo image may include words, letters, numbers, symbols, graphics, and combinations thereof.

The tattoo transfer devices described herein unexpectedly provide a way to precisely apply a semi-permanent tattoo image on the skin of an human or animal subject. In contrast with temporary tattoo images, which typically sit like a sticker or label on top of the subject's skin, and therefore are relatively easily removed and typically last for only a few days, the semi-permanent tattoo images described herein develop within several layers of the subject's skin, and therefore are not easily removed and typically last for one to two weeks. Further, the devices, compositions, and methods described herein unexpectedly provide a way for a semi-permanent tattoo image to be applied, so that once developed on the subject's skin, the tattoo image is clear and crisp. The devices are used to create a desired image on a human or animal subject's skin, wherein the image is not necessarily immediately present on the skin yet develops after a period of time. Development of the desired image on the subject's skin occurs due to interactions between the skin or components of the skin and at least one component within the image-forming composition or compositions.

The devices described herein apply an image-forming composition layer or film, referred to herein as an “image-forming composition layer”, onto a subject's skin. The image-forming composition layer applied onto the subject's skin according to the invention is typically in the range of about 25-85 μm (microns), preferably 40-50 μm (microns), more preferably about 45 μm (microns), and even more preferably about 44 μm (microns). Once applied to the subject's skin, the image-forming composition layer is comfortable to the subject, due to the flexibility and thinness of the layer. The subject may even forget that the layer has been applied. The image-forming composition layer is not removed once it is applied to the subject's skin. Instead, the layer will eventually slough off with normal skin cell turnover of the subject's skin.

The device is produced by placing onto a base sheet an image-forming composition layer, wherein the composition has been placed in a design corresponding to the desired tattoo image to be formed on the subject's skin. The image-forming composition is comprised of a combination of image-forming components and adhering components, and the image-forming composition layer forms a bond with the subject's skin at the area on the subject's skin where the tattoo image is desired to be applied. The bond holds the image-forming composition layer in place on the subject's skin, and the image-forming component in the image-forming composition layer reacts with, or dyes, or both reacts with and dyes, the subject's skin, thereby resulting in a crisp, clearly defined tattoo image on the subject's skin, without smudging or blurred images.

The term “semi-permanent” as used herein means that once the tattoo image is applied to a subject's skin, the tattoo image cannot be easily rubbed off or washed off the subject's skin for a time period ranging from days to weeks. In preferred embodiments, the semi-permanent tattoo image remains on the subject's skin for at least about one week, and preferably about 2 or more weeks.

In one embodiment, the semi-permanent tattoo image does not appear (i.e., does not become substantially visible to the naked eye) on the subject's skin immediately after the image-forming composition is applied to the subject's skin using the device. Instead, the tattoo image develops over a period of time after the image-forming composition is applied to the subject's skin. The term “develops” as used herein means that the tattoo image becomes increasingly visible and darkens over time on the subject's skin. In other words, the tattoo image on the subject's skin becomes more saturated or opaque over time, until it reaches its maximum saturation or opaqueness.

In a preferred embodiment, the development of the tattoo image occurs solely or primarily as a result of chemical interactions between substances naturally in a subject's skin and the image-forming composition. It takes a period of time, typically ranging from several hours to several days, for the tattoo image to develop on and within the top layers of the subject's skin, so that the tattoo image gradually becomes visible. Typically, it takes about 24-48 hours for the tattoo image to fully develop on the subject's skin, meaning that the image has reached its maximum darkness or opacity. When full-developed, the tattoo image typically appears as dark purple, blue or black on the skin. The fully-developed tattoo image fades slowly over the course of about 2 to 3 weeks.

The tattoo device is preferably comprised of at least two layers, as described herein. At least one of the layers is comprised of an image-forming composition. The image-forming composition layer is typically formed on a first side of a base layer, but does not cover the entire first side of the base layer. The image-forming composition layer corresponds with the design of the eventual tattoo image that will appear on the subject's skin. For example, if the desired tattoo image for the subject's skin is an outline of a heart symbol above the word “LOVE”, then the image-forming composition layer is formed on the base layer as an outline of a heart symbol above the word “LOVE” but in reverse to the desired tattoo image. The image-forming layer is typically non-continuous, because the layer is formed in the design of the desired eventual tattoo image.

In one embodiment, the image-forming composition appears colorless or substantially colorless on the tattoo device.

In yet another embodiment, the image-forming composition contains a visibility tint or other ingredient or component that renders the image-forming composition layer visible on the tattoo device before it is applied to the subject's skin, thereby rendering visible the design of the tattoo image that will develop as a result of use of the device.

In still yet another embodiment, the image-forming composition contains a visibility tint or other ingredient or component that renders the image-forming composition layer visible on the subject's skin substantially immediately after the layer is applied to the subject's skin, thereby providing visible confirmation of the orientation and placement of the tattoo image that will eventually develop.

In a preferred embodiment, the image-forming composition layer is visible both when it is on the tattoo device and also substantially immediately after the layer is applied to the subject's skin.

Although the semi-permanent tattoo devices described herein are primarily intended for use to apply semi-permanent tattoo images to the skin of a human, it is possible for the devices to be used to apply semi-permanent tattoo images to the skin of an animal. If the animal or human subject's skin where the tattoo image is to be applied contains fur or hair, it is preferable that the fur or hair is removed prior to using the tattoo device.

The image-forming composition in its most basic form comprises at least one image-forming component that forms a semi-permanent tattoo image on the subject's skin, and at least one adhering component capable of adhering to the subject's skin. The image-forming composition may further comprise other components.

The term “component” as used in this disclosure includes compositions, mixtures, extracts, substances, and compounds.

In a preferred embodiment, the image-forming composition comprises at least one image-forming component that reacts with the subject's skin to cause the development of a semi-permanent tattoo image on (or within the top layers of) the subject's skin, and at least one adhering component capable of adhering to the subject's skin.

In yet another embodiment, the image-forming composition may comprise at least one coloring component that stains, colors, or dyes (i.e., changes the color of) the subject's skin to form a semi-permanent tattoo image on the subject's skin immediately or substantially immediately after it is applied to the skin, and at least one adhering component capable of adhering to the subject's skin.

In still yet another alternative embodiment, the image-forming composition may comprise at least one image-forming component that reacts with the subject's skin to cause the development of a semi-permanent tattoo image on the subject's skin; at least one coloring component that stains, colors, or dyes (i.e., changes the color of) the subject's skin and is capable of forming a semi-permanent tattoo image on the subject's skin; and at least one adhering component capable of adhering to the subject's skin.

In certain exemplary embodiments, the image-forming composition comprises an image-forming component that reacts with the subject's skin to cause the development of a semi-permanent tattoo image on the subject's skin, wherein the image-forming component is selected from extracts of the Rubiaceae family. More particularly, extracts of plants of the Genipa genera are preferred. These extracts are referred to herein as “Genipin”.

Genipin is preferred to be used. More preferably, the Genipin is in powder form and is of high purity. In a preferred embodiment, the Genipin has a purity of at least about 95%, although Genipin with a lower purity may be used instead.

In another embodiment, the image-forming composition comprises as the image-forming component Genipin obtained from sources such as Genipa americana fruit extract or Gardenia jasminoides extract, but may be obtained from other sources. Derivatives of Genipin obtained by chemical modification of Genipin, of Genipa americana fruit extract and/or Gardenia jasminoides extract may be used in place of Genipin or in addition to Genipin, provided that the derivatives react with the subject's skin, resulting in development of a semi-permanent tattoo image on the subject's skin.

In embodiments of the image-forming composition that comprise one or more coloring components that simply stain, color or dye (i.e., change the color of) the subject's skin substantially immediately after the image-forming composition is applied to the subject's skin, examples of coloring components include but are not limited to plant-derived or food-derived extracts and other substances, provided that they are skin-safe. For example, all or a portion of the Genipin may be replaced with a dye such as Henna, or with other skin-safe dyes or other substances capable of staining the skin. Other nonlimiting examples of such components are turmeric, beet juice, concentrated beet juice, red or purple grape juice, and concentrated grape juice. Any component capable of staining the skin may be used, provided that the component is generally recognized as safe to apply to the skin.

The adhering component in the image-forming composition that adheres to the subject's skin should be a skin-safe component with adhesive properties. The image-forming composition will preferably have a good balance of peel and tack properties, high cohesive strength, and be capable of forming a bond with the subject's skin.

The adhering skin-safe component with adhesive properties should be a skin-safe adhesive. Nonlimiting examples of skin-safe components with adhesive properties and adhesives that may be used include acrylates, acrylate co-polymers, and vinyl-acrylate co-polymers, and mixtures thereof. Preferred examples of a skin-safe component with adhesive properties are pressure sensitive adhesives and permanent pressure sensitive adhesives.

Preferably, the skin-safe component with adhesive properties will have a good balance of peel and tack properties, high cohesive strength, have excellent adhesion to human skin, and can be transfer coated on most rod, gravure or roll coaters and the like. Even more preferably, the skin-safe component with adhesive properties is formaldehyde-free and meets USFDA requirements for adhesives and adhesive components used in contact with skin.

The skin-safe component with adhesive properties permits the image-forming composition layer to form a bond with the subject's skin at the area on the subject's skin where the tattoo image is desired to be applied. The bond should be sufficiently strong to prevent movement of the image-forming composition layer across the surface of the subject's skin, yet the bond with the skin should be capable of being broken so that the tattoo device can be removed if necessary, with little to no discomfort or skin damage to the subject. The bond retains the image-forming composition in place, thereby substantially or completely avoiding movement of the image-forming composition layer from where it is initially placed. Retaining the image-forming composition layer in place on the subject's skin, without moving the layer across the subject's skin, helps to ensure that the resulting tattoo image will be clear (register correctly). If the image-forming composition layer is not maintained in place on the subject's skin, it may result in a tattoo image that is unclear, smeared or otherwise lacking clarity or darkness.

In certain embodiments, the skin-safe component with adhesive properties may be comprised, in whole or in part, of natural gums and/or natural rosins. Without limiting the foregoing, gums and rosins may be selected from pine gum rosin, guar gum, and mixtures of two or more of the foregoing. Other examples of gums and rosins that maybe used are pentaerythrityl hydrogenated rosinate, glyceryl hydrogenated rosinate, and pine rosin, glyceryl rosinate, and mixtures thereof.

Mixtures or combinations of one or more different skin-safe components having adhesive properties may also be used in the image-forming composition, provided that the image-forming composition has a good balance of peel and tack properties, high cohesive strength, and is capable of forming a bond with the subject's skin.

It is preferable that the image-forming composition is capable of being transfer coated on most rod, gravure or roll coaters and the like.

In preferred embodiments of a device or structure according to the invention, the device or structure is made using an image-forming composition comprised of Genipin, which is a skin-safe image-forming component, a skin-safe adhering component with adhesive properties, and a skin-safe oil. In addition or instead of the skin-safe oil, a skin-safe emollient may be used.

Nonlimiting examples of skin-safe oils that may be used are vegetable oils, isolates of vegetable oils, non-toxic essential oils, non-toxic isolates of essential oils, and/or mixtures of two or more of the foregoing. Without limiting the foregoing, specific examples of skin-safe oils that may be used are Vitamin E, tocopherol, D-limonene, pinene, squalene, and mixtures thereof.

An alternative to using a skin-safe oil is to replace all or a portion of the oil with a component or substance that acts as a skin moisturizer and/or emollient, wherein cetyl alcohol is just one non-limiting example.

In yet another embodiment the image-forming composition optionally further comprises at least one visibility component that renders a facsimile of the tattoo image visible to naked eye. This is especially useful if the image-forming component, and the image-forming composition, are not visible unless the image-forming component has had an extended time to react with a subject's skin. For example, a visibility tint may be used to permit the user of the tattoo device or the subject to visualize the tattoo image on the subject's skin immediately after the tattoo image is applied to the subject's skin, before the image actually appears or develops on the subject's skin.

The term “user” as used herein refers to a human subject who uses the device to apply the tattoo image to the subject's own skin, and also refers to a person who uses the device to apply the tattoo image to the skin of a human subject other than that person, or to the skin of an animal subject.

In yet another embodiment, the image-forming composition optionally further comprises a visibility component, whose function is to make the image visible on the tattoo device, prior to the tattoo being applied to the subject's skin. Having the image visible on the tattoo device may assist a user to place it on the subject's skin in the desired orientation. An example of a visibility component that may be used are various visibility tints. Preferably, the visibility tint is in dry powder form when it is added to the image-forming composition.

The image-forming composition may further comprise a solvent. Preferably the solvent is capable of dissolving Genipin. Nonlimiting examples of solvents include alcohol or other solvent selected from ethanol, isopropyl alcohol and dimethyl sulfoxide (DMSO), and mixtures of two or more of the foregoing.

In an embodiment of a device according to the invention, the device is made using an image-forming composition that comprises: Genipin, an acrylate-containing skin-safe adhesive, and a skin-safe oil.

In yet another embodiment, the image-forming composition is comprised of:
about 85-98 wt % skin-safe component with adhesive properties;
about 2-7 wt % skin-safe oil; and
about 0.5-4 wt % Genipin.

In still yet another embodiment, the image-forming composition is comprised of:
about 85-98 wt % skin-safe acrylate;
about 2-7 wt % Vitamin E oil; and
about 0.5-4 wt % Genipin.

In still yet another embodiment, the image-forming composition is comprised of Genipin, acrylate adhesive, Vitamin E oil, and ethanol. The ethanol acts as a solvent for the Genipin.

Yet another embodiment of the image-forming composition is comprised of Genipa americana fruit extract, natural pine gum rosin, limonene, and isopropyl alcohol.

Another alternative embodiment of the image-forming composition is comprised of Gardenia jasminoides extract, guar gum, pinene, and dimethyl sulfoxide (DMSO).

The following is a non-limiting example of a process for producing the image-forming composition, and using the composition to form a tattoo device. The ingredients of the composition are mixed together, then the composition is “aged” for a few days (typically about 48-72 hours). During the aging, any water and organic solvent in the mixture will substantially evaporate out.

The image-forming composition may then be used to form a tattoo device, such as by applying the composition by screen printing/offset press/lithography onto a base layer or release liner. The device then is treated in drying oven or by other means to cause substantially all of any water and solvent present in the device to evaporate off.

The tattoo device may be produced in whole or part using one of more of the following methods: screen printing, flexographic printing, offset printing, gravure printing, lithography, ink jet printing, or stenciling. In particular, one or more of these methods (or other methods known in the industry) could be used to apply the image-forming composition to a backing sheet.

An example of an embodiment of a semi-permanent tattoo device according to the invention includes:
(a) a base sheet having a first surface and a second surface; and
(b) an image-forming composition layer having a first surface and a second surface, wherein the layer is applied, in the design, pattern, or shape of the desired eventual tattoo image, onto the second surface of the base sheet so that the first surface of the image-forming composition layer is formed on and is adjacent to the second surface of the base sheet.
   Optionally, the device may further include a substantially transparent protective liner sheet provided on the second surface of the image-forming composition layer, to protect the image-forming composition layer, wherein the protective liner sheet is removable for exposing the image-forming composition layer which will be placed in contact with a subject's skin. It is preferred, but not necessary, that the protective liner sheet is substantially transparent.

The term “base sheet” as used herein may be a paper sheet or sturdy synthetic film or sheet. The base sheet preferably has a thickness in the range of about 0.0004 to about 0.012 inches, i.e., between about 4 pt and 12 pt. Other thicknesses are possible. The base sheet may be a paper sheet having a starch base.

The general method for using the tattoo device comprising a base sheet and an image-forming composition layer is as follows: the second surface of the image-forming composition layer is placed in contact with the subject's skin, in the desired location on the subject's skin, and the base sheet is removed from the image-forming composition layer, thereby leaving the image-forming composition layer in contact with and substantially adhered to the subject's skin. Preferably, prior to removing the base sheet, the user or subject applies pressure to the first surface of the base sheet for at least about sixty (60) seconds. Even more preferably, prior to removing the base sheet, the user or subject covers the base sheet with a damp cloth, and applies firm pressure evenly against the damp cloth that is across the entire base sheet for at least about sixty (60) seconds.

Once the base sheet has been removed, the image-forming composition layer remains in contact with and adhered to the subject's skin. The flexible image-forming composition layer on the subject's skin looks and feel unobtrusive to the subject, due to the layer's flexibility and thinness. In some embodiments of the devices, the image-forming composition layer is substantially transparent and color-free, thereby making the layer difficult to see on the subject's skin.

In other embodiments of the semi-permanent tattoo devices according to the invention, the second surface of the base sheet has or is provided with a coating of a release agent, to assist the user or subject to separate or release the image-forming composition layer from the base sheet. For example, the release agent may be one that releases upon exposure to water, pressure or heat, or to a combination of two or more of water, pressure or heat. Non-limiting examples of potential release agents are starches, dextrin, silicone, and combinations thereof, although any release agent known to those of skill in the arts of temporary tattoos and semi-permanent tattoos may be employed.

Other embodiments of the semi-permanent tattoo devices according to the invention may contain additional layers and coatings. For example, the devices may have a substantially transparent precoating may be provided between the base sheet and the image-forming composition layer. This precoating is preferably colorless, so as not to obscure the visible components, if any, of the image-forming composition layer, and so that precoating is not visible or is minimally visible when it is deployed on the subject's skin. The precoating should be a skin-safe composition, and is typically applied to the second side of the base sheet prior to providing the base sheet with the image forming composition layer. If the second surface of the base sheet has a coating of a release agent, then the precoating is applied over the release agent.

The precoating serves a protective function, by providing a barrier between the image-forming composition layer on the tattoo device and the environment, once the semi-permanent tattoo image is transferred to the subject's skin and the base sheet is removed. When the tattoo device is used to deploy the image-forming composition onto the subject's skin, the base sheet will be discarded, leaving behind the image-forming composition layer in a design corresponding to the desired tattoo image. The precoating, if used, preferably will remain integral with the image-forming composition layer after application to the subject's skin, to further increase the wear-resistance of the image-forming composition layer on the subject's skin, thereby increasing the period of time during which the image can develop on the subject's skin or in the layers of the subject's skin and by increasing the wear-resistance of the tattoo image on the skin.

The precoating may be applied in any manner known to one of skill in the art, such as via screen, gravure or offset printing technologies. In an embodiment, the precoating is applied using a printing unit.

Optionally, instead of or in addition to a precoating, a substantially transparent integrity-preserving film may be used between the backing and the image-forming composition layer; when the image-forming composition layer is deployed onto the subject's skin, the integrity-preserving film will remain over and protect the image-forming composition layer deployed onto the subject's skin. The integrity-preserving film preserves the integrity of the image-forming composition layer, until the tattoo image develops on the subject's skin. The integrity-preserving film may be substantially comprised of silicone or a silicone copolymer, for example.

Alternatively, or in addition to a precoating and/or an integrity-preserving film, the user may apply, on top of the image-forming composition layer that has been deployed on the subject's skin, a protective film or other sheet that is not part of the tattoo device, to prevent or substantially minimize movement across the subject's skin of the image-forming composition layer. The protective film may optionally be provided to the user along with the tattoo device. If the protective film or other sheet is used, a preferred example is soft silicone tape, similar or the same as that used to cover IV ports in skin.

Optionally, a protective liner, film, sheet, or coating, collectively referred to herein as “protective liner” may be applied to the tattoo device, on top of the second surface of the image-forming composition layer or other layers, if present. The purpose of the protective liner is to prevent the image-forming composition layer from adhering to the subject's skin and other surfaces, until it is desired to use the tattoo device. The protective liner may be used to prevent multiple tattoo devices from adhering to one another, and to prevent the tattoo device, especially the image-forming composition layer, from being damaged during packaging, shipping, and other times before it is applied to the subject's skin. The protective liner is preferably substantially transparent and colorless, so that it does not obscure the view of the image-forming composition layer before it is applied to the subject's skin. Most preferably, the protective liner sheet is clear. The protective liner is designed to be removable by the user or subject, preferably immediately prior to using the tattoo device. The protective liner can be made from a variety of materials, such as polyethylene or polypropylene. It is important that the protective liner remain in place on the tattoo device until it is intentionally removed by the user or subject, and that when removed, the liner should not damage the image-forming composition layer or other layers or coatings thereon.

When the tattoo devices are manufactured, multiple devices may be produced simultaneously, and then cut into individual tattoo devices or into smaller sheets comprising multiple tattoo devices. Preferably, cutting is performed after the protective liner sheet is applied to the image-forming composition layer.

The tattoo device describe herein may be used in contact with human skin, and therefore it is very important that the adhesives and inks and its other components are skin safe, preferably cosmetic grade, meaning that they are unlikely to cause rashes or other reactions when applied to the subject's skin.

The semi-permanent tattoo image as described herein is applied to the subject's skin as follows. If present, the protective liner sheet is removed, such as by peeling it off, thereby exposing or uncovering the image-forming composition layer. Immediately thereafter, the tattoo is placed on the desired position on the subject's skin, with the image-forming composition layer in direct contact with the subject's skin. Next, heat, pressure, or moisture, or a combination of one or more of heat, pressure, or moisture, is applied to the base sheet. Then, the base sheet is removed or peeled off the image-forming composition layer. If the image-forming composition layer contains a visibility tint, then a precursor to the semi-permanent tattoo image will be visible. As used herein, “precursor to the semi-permanent tattoo image” means an image substantially similar to the design (e.g., lettering, numbers, symbols, graphics, etc.) of the eventual semi-permanent tattoo image that will develop on or in the subject's skin.

The image-forming composition layer (including any remaining remnants of the precoating or release coating from the base layer, as well as the integrity-preserving film, if used), will wear off gradually, leaving a semi-permanent tattoo image developed on or within the top layers of the subject's skin.

1. A semi-permanent tattoo transfer device, for forming a semi-permanent tattoo image on a human or animal subject's skin, comprising a base sheet and an image-forming layer corresponding to a design of the semi-permanent tattoo image, wherein the image-forming layer comprises:
a. a skin-safe image-forming component; and
b. a skin-safe adhering component.

2. The semi-permanent tattoo transfer device of claim 1, wherein the skin-safe image-forming component is capable of reacting with the subject's skin to cause development of a semi-permanent tattoo image on the subject's skin.

3. The semi-permanent tattoo transfer device of claim 1, wherein the skin-safe image-forming component is capable of dyeing or coloring or staining the subject's skin substantially immediately upon contact.

4. The semi-permanent tattoo transfer device of claim 2, wherein the skin-safe image-forming component is Genipin.

5. The semi-permanent tattoo transfer device of claim 4, wherein the Genipin is in powder form.

6. The semi-permanent tattoo transfer device of claim 4, wherein the Genipin has a purity of at least about 95%.

7. The semi-permanent tattoo transfer device of claim 4, wherein the image forming layer is formed from a composition comprising Genipin in an amount of about 0.5-4 wt % of the composition.

8. The semi-permanent tattoo transfer device of claim 4, wherein the Genipin is obtained from an extract of Genipa americana fruit or Gardenia jasminoides.

9. The semi-permanent tattoo transfer device of claim 2, wherein the skin-safe image-forming component is a derivative of Genipin.

10. The semi-permanent tattoo transfer device of claim 2, wherein the skin-safe adhering component is a pressure sensitive adhesive.

11. The semi-permanent tattoo transfer device of claim 2, wherein the skin-safe adhering component is selected from the group consisting of an acrylate adhesive, an acrylate co-polymer adhesive, a vinyl-acrylate co-polymer adhesive, and combinations thereof.

12. The semi-permanent tattoo transfer device of claim 2, wherein the image-forming layer is formed from a composition that further comprises at least one oil or emollient.

13. The semi-permanent tattoo transfer device of claim 12, wherein the oil or emollient is selected from the group consisting of vegetable oil, an isolate of vegetable oil, a non-toxic essential oil, a non-toxic isolate of an essential oil, and combinations thereof.

14. The semi-permanent tattoo transfer device of claim 13, wherein the oil or emollient is selected from the group consisting of Vitamin E, tocopherol, D-limonene, pinene, squalene, cetyl alcohol, and combinations thereof.

15. The semi-permanent tattoo transfer device of claim 2, wherein the image-forming layer further comprises a visibility tint.

16. The semi-permanent tattoo transfer device of claim 3, wherein the skin-safe image forming component is selected from the group consisting of henna, turmeric, beet juice, concentrated beet juice, grape juice, concentrated grape juice, and combinations thereof.

17. A semi-permanent tattoo transfer device for forming, on a human or animal subject's skin, a semi-permanent tattoo image, comprising:
a. a base sheet having a first surface and a second surface; and
b. an image-forming layer on the second surface of the base sheet, wherein the image-forming layer is comprised of
a skin-safe image-forming component selected from Genipin, a derivative of Genipin, and combinations thereof,
a skin-safe adhering component, and
a skin-safe oil or emollient,
wherein the image-forming composition layer is in a design, pattern, or shape corresponding to the semi-permanent tattoo image.

18. The semi-permanent tattoo transfer device of claim 17, wherein the image-forming layer is formed of a composition comprising:
a. about 0.5-4 wt % of Genipin as the skin-safe image-forming component;
b. about 85-98 wt % of skin-safe adhering component; and
c. about 2-7 wt % of at least one skin-safe oil or emollient.

19. The semi-permanent tattoo transfer device of claim 18, wherein the image-forming layer has a thickness in the range of about 25 to about 65 microns.

20. The semi-permanent tattoo transfer device of claim 18, wherein the image-forming layer comprises:
a first surface adjacent the second surface of the base sheet, and
a second surface opposite the first surface of the image-forming layer,
and further comprising a protective liner on the second surface of the image-forming layer.

21. The semi-permanent tattoo transfer device of claim 20, wherein the protective liner is substantially transparent.

22. The semi-permanent tattoo transfer device of claim 1, wherein the image-forming layer is formed by a process selected from the group consisting of:
screen printing, flexographic printing, offset printing, gravure printing, lithography, ink jet printing, and stenciling.

23. (canceled)

24. (canceled)

25. The device of claim 18, wherein the image-forming layer is formed from a composition comprising:
a. about 0.5-4 wt % of Genipin;
b. about 85-98 wt % of skin-safe acrylate adhesive; and
c. about 2-7 wt % of Vitamin E oil.

26. The device of claim 17, further comprising a release agent on the second surface of the base sheet.

27. The device of claim 17, further comprising a precoating or an integrity-preserving film between the second surface of the base sheet and the image-forming layer.
